(19) United States
(12) Patent Application Publication
(10) Pub. No.: US 2025/0228713 A1
(43) Pub. Date: Jul. 17, 2025

(54) METHOD AND APPARATUS FOR MANUFACTURING DISPOSABLE WEAR

(71) Applicant: ZUIKO CORPORATION, Ibaraki-shi, Osaka (JP)

(72) Inventor: Hirofumi YAMAUCHI, Ibaraki-shi, Osaka (JP)

(21) Appl. No.: 18/853,719

(22) PCT Filed: Apr. 19, 2023
(86) PCT No.: PCT/JP2023/015679

§ 371 Date: Oct. 3, 2024

(30) Foreign Application Priority Data
Apr. 27, 2022 (JP) 2022-073573

(51) Int. Cl. A61F 13/15

(52) U.S. Cl. CPC A61F 13/15723; A61F 13/15739; A61F 13/15747; A61F 13/15764; A61F 13/15804

(57) ABSTRACT

A method of manufacturing a disposable wear includes conveying, in a first direction, an absorbent-body continuous body in which first portions to be made into absorbent bodies are connected; and conveying, in a second direction, a waist-member continuous body in which second portions to be made into waist members are connected. The method includes forming the absorbent bodies by cutting the absorbent-body continuous body; and forming folded absorbent bodies by folding the absorbent bodies into two parts. The method includes rotating the folded absorbent bodies by 90 degrees; and forming a wear continuous body by placing, onto the waist-member continuous body, the folded absorbent bodies, which have been rotated by 90 degrees, apart from each other, and bonding the folded absorbent bodies to the waist-member continuous body. The method includes forming disposable wears by cutting the wear continuous body at cutting positions located between adjacent the absorbent bodies.

### TECHNICAL FIELD

The present invention relates to a method and an apparatus for manufacturing a disposable wear, and more specifically, relates to a technique for manufacturing a substantially T-shaped disposable wear.

### BACKGROUND ART

For example, FIG. 4 is a developed view of a substantially T-shaped disposable wear 101. As shown in FIG. 4, in the disposable wear 101, one end side of an absorbent body 102 is bonded to an intermediate position of a waist member 103. The disposable wear 101 is provided in a state where the other end side 102c of the absorbent body 102 is folded back at a central part 102b toward the waist member 103 so that the absorbent body 102 is folded into two parts and the waist member is folded so that both end parts overlap.

FIG. 5 is a perspective view showing a manufacturing process of the disposable wear 101. As shown in FIG. 5, a wear continuous body 107 in which a plurality of absorbent bodies 102 have one end sides thereof bonded in a comb shape with a predetermined spacing therebetween is formed while a waist-member continuous body 171 in which portions to be made into waist members 103 are connected is conveyed in the direction indicated by the arrow Y. Then, while the wear continuous body 107 is being conveyed, the waist-member continuous body 171 side of the wear continuous body 107 is lifted and placed on the other end sides 102c of the absorbent bodies 102 to fold the absorbent bodies 102 into two parts so that fold lines 126 are formed at intermediate positions of the absorbent bodies 102 in a direction X orthogonal to the conveyance direction Y. Then, the wear continuous body 107 is cut between the adjoining absorbent bodies 102 to form disposable wears.

### CITATION LIST

### Patent Literature

[Patent Literature 1] JP 4920424 B2

### SUMMARY OF THE INVENTION

### Problem to be Solved by the Invention

In a case where the absorbent bodies are folded into two parts while the wear continuous body is being conveyed as described above, the absorbent bodies 102 are folded into two parts with the angle being gradually increased so that no excessive force acts on the absorbent bodies 102. For this reason, the section where the absorbent bodies 102 are to be folded into two parts is long, which increases the installation space of the disposable wear manufacturing apparatus.

In view of such circumstances, a problem to be solved by the present invention is to provide a method and an apparatus for manufacturing a disposable wear with which it is possible to shorten the section where the absorbent bodies are to be folded into two parts.

### Means for Solving the Problem

To solve the above-mentioned problem, the present invention provides a disposable wear manufacturing method structured as follows:

The disposable wear manufacturing method is provided with: (i) a first conveying step of conveying, in a first direction, an absorbent-body continuous body in which first portions to be made into absorbent bodies are connected in the first direction; (ii) a second conveying step of conveying, in a second direction, a waist-member continuous body in which second portions to be made into waist members are connected in the second direction; (iii) a first cutting step of forming the absorbent bodies by cutting the absorbent-body continuous body being conveyed in the first direction; (iv) a folding step of forming folded absorbent bodies by folding the cut absorbent bodies into two parts so that fold lines are formed in a direction orthogonal to the first direction while conveying the cut absorbent bodies along a cylindrical conveyance path; (v) a rotating step of rotating the folded absorbent bodies by 90 degrees while conveying the folded absorbent bodies; (vi) a bonding step of forming a wear continuous body by placing more than one of the 90-degree rotated absorbent bodies on the waist-member continuous body being conveyed in the second direction, with a spacing therebetween, and bonding the absorbent bodies to the waist-member continuous body; and (vii) a second cutting step of forming disposable wears in each of which the folded absorbent body is bonded to an intermediate position of the waist member, by cutting the wear continuous body at cutting positions between the adjoining absorbent bodies.

According to the above-described method, since the absorbent bodies are folded into two parts while being conveyed along the cylindrical conveyance path, the section where the absorbent bodies are to be folded into two parts can be made short compared with when the absorbent bodies are folded into two parts while being conveyed along a linear conveyance path.

Preferably, the disposable wear manufacturing method is further provided with (viii) a temporarily fixing step of temporarily fixing together a first region and a second region divided by the fold line of each of the folded absorbent bodies.

In this case, the temporarily fixed folded absorbent bodies can be conveyed at higher speed because the folded state is stabilized, so that the number of disposable wears manufactured per unit time can be increased.

Preferably, the disposable wear manufacturing method is further provided with (ix) a first repitching step where by using a first moving pad, the folded absorbent bodies are received from the folding step, the received folded absorbent bodies are transferred to the rotating step, and the first moving pad receives the folded absorbent bodies from the folding step while moving at a first reception speed and transfers the received folded absorbent bodies to the rotating step while moving at a first transfer speed different from the first reception speed.

In this case, it is possible to accommodate size differences of the absorbent bodies.

Preferably, the disposable wear manufacturing method is further provided with (x) a second repitching step where by using a second moving pad, the 90-degree rotated folded absorbent bodies are received from the rotating step, the received folded absorbent bodies are transferred to the bonding step, and the second moving pad receives the 90-degree rotated folded absorbent bodies from the rotating step while moving at a second reception speed and transfers the received folded absorbent bodies to the bonding step while moving at a second transfer speed different from the second reception speed.

In this case, it is possible to accommodate size differences of the waist members.

Preferably, the disposable wear manufacturing method is further provided with: (ix) a first repitching step where by using a first moving pad, the folded absorbent bodies are received from the folding step, the received folded absorbent bodies are transferred to the rotating step, and the first moving pad receives the folded absorbent bodies from the folding step while moving at a first reception speed and transfers the received folded absorbent bodies to the rotating step while moving at a first transfer speed different from the first reception speed; and (x) a second repitching step where by using a second moving pad, the 90-degree rotated folded absorbent bodies are received from the rotating step, the received folded absorbent bodies are transferred to the bonding step, and the second moving pad receives the 90-degree rotated folded absorbent bodies from the rotating step while moving at a second reception speed and transfers the received folded absorbent bodies to the bonding step while moving at a second transfer speed different from the second reception speed.

In this case, even if the sizes of both of the absorbent bodies and the waist members change according to the size of the disposable wears, it is possible to accommodate size differences of the disposable wears.

Moreover, to solve the above-mentioned problem, the present invention provides a disposable wear manufacturing apparatus structured as follows:

The disposable wear manufacturing apparatus is provided with: (a) a first unit that forms absorbent bodies by cutting an absorbent-body continuous body in which first portions to be made into the absorbent bodies are connected in a first direction, while conveying the absorbent-body continuous body in the first direction, and forms folded absorbent bodies by folding the absorbent bodies into two parts so that fold lines are formed in a direction orthogonal to a conveyance direction of the absorbent bodies, while conveying the absorbent bodies along a cylindrical conveyance path: (b) a rotating unit that rotates the folded absorbent bodies by 90 degrees while conveying the folded absorbent bodies: and (c) a second unit that forms a wear continuous body by placing more than one of the 90-degree rotated folded absorbent bodies on a waist member continuous body in which second portions to be made into waist members are connected in a second direction, with a spacing therebetween while conveying the waist member continuous body in the second direction and bonding the folded absorbent bodies to the waist-member continuous body, and forms disposable wears in each of which the folded absorbent body is bonded to an intermediate position of the waist member, by cutting the wear continuous body at cutting positions between the adjoining absorbent bodies.

According to the above-described structure, since the absorbent bodies are folded into two parts while being conveyed along the cylindrical conveyance path, the section where the absorbent bodies are to be folded into two parts can be made short compared with when the absorbent bodies are folded into two parts while being conveyed along a linear conveyance path.

Preferably, the disposable wear manufacturing apparatus is further provided with: (d) a temporarily fixing device that temporarily fixes together a first region and a second region divided by the fold line of each of the folded absorbent bodies.

In this case, the folded absorbent bodies can be conveyed at higher speed because the folded state of the absorbent bodies is stabilized, so that the number of disposable wears manufactured per unit time can be increased.

Preferably, the disposable wear manufacturing apparatus is further provided with: (e) a first repitching unit having a first moving pad that receives the folded absorbent bodies from the first unit while moving at a first reception speed and transfers the received folded absorbent bodies to the rotating unit while moving at a first transfer speed different from the first reception speed.

In this case, it is possible to accommodate size differences of the absorbent bodies.

Preferably, the disposable wear manufacturing apparatus is further provided with: (f) a second repitching unit having a second moving pad that receives the 90-degree rotated folded absorbent bodies from the rotating unit while moving at a second reception speed and transfers the received absorbent bodies to the second unit while moving at a second transfer speed different from the second reception speed.

In this case, it is possible to accommodate size differences of the waist members.

Preferably, the disposable wear manufacturing apparatus is further provided with: (g) a first repitching unit having a first moving pad that receives the folded absorbent bodies from the first unit while moving at a first reception speed and transfers the received folded absorbent bodies to the rotating unit while moving at a first transfer speed different from the first reception speed; and: (f) a second repitching unit having a second moving pad that receives the 90-degree rotated folded absorbent bodies from the rotating unit while moving at a second reception speed and transfers the received absorbent bodies to the second unit while moving at a second transfer speed different from the second reception speed.

In this case, even if the sizes of both of the absorbent bodies and the waist members change according to the size of the disposable wears, it is possible to accommodate size differences of the disposable wears.

### Effects of the Invention

According to the present invention, it is possible to shorten the section where the absorbent bodies are to be folded into two parts.

### BRIEF DESCRIPTION OF THE DRAWINGS

FIG. 1 is a schematic view of a disposable wear manufacturing apparatus (first embodiment).

FIG. 2 is a schematic view of the disposable wear manufacturing apparatus (first embodiment).

FIG. 3 is an explanatory view of a disposable wear manufacturing process (first embodiment).

FIG. 4 is a developed view of the disposable wear (first conventional example).

FIG. 5 is a perspective view showing the manufacturing process of the disposable wear (first conventional example).

### MODE FOR CARRYING OUT THE INVENTION

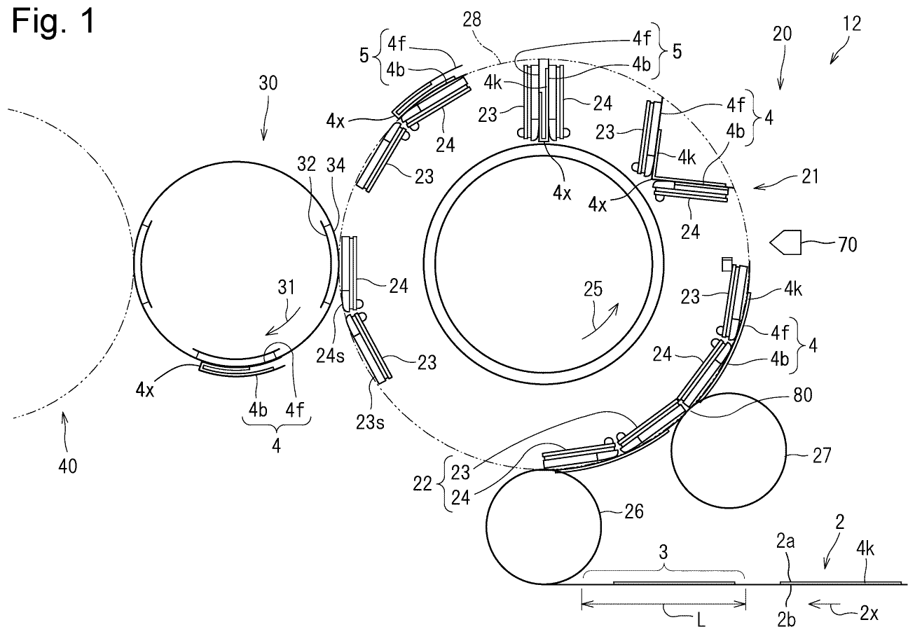

Hereinafter, an embodiment of the present invention will be described with reference to the drawings.

[First embodiment] A disposable wear manufacturing method and apparatus of a first embodiment will be described with reference to FIGS. 1 to 3.

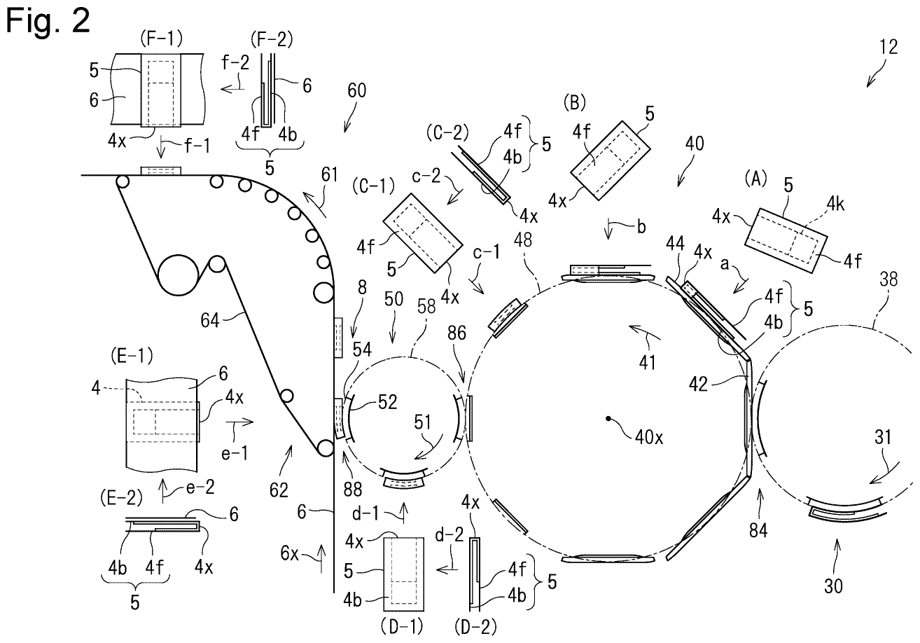

FIGS. 1 and 2 are schematic views of a disposable wear manufacturing apparatus 12. In FIG. 2, (A), (B), (C-1), (C-2), (D-1), (D-2), (E-1), (E-2), (F-1) and (F-2) are schematic views from the directions indicated by the arrows a, b, c-1, c-2, d-1, d-2, e-1, e-2, f-1 and f-2, respectively.

As shown in FIGS. 1 and 2, the disposable wear manufacturing apparatus 12 is provided with a first unit 20, a first repitching unit 30, a rotating unit 40, a second repitching unit 50, a second unit 60 and a temporarily fixing device 70.

In the first unit 20, a guide roll 26 and a cutter roll 27 are disposed adjacent to a folding drum 21. The folding drum 21 conveys an absorbent-body continuous body 2 fed through the guide roll 26, along a cylindrical conveyance path 28. The cutter roll 27 cuts the absorbent-body continuous body 2 being conveyed by the folding drum 21, thereby forming absorbent bodies 4. The folding drum 21 folds the absorbent bodies 4 into two parts while conveying them along the cylindrical conveyance path 28, thereby forming folded absorbent bodies 5.

In the absorbent-body continuous body 2, first portions 3 having a predetermined length L and to be made into the absorbent bodies 4 are connected in a first direction 2x indicated by the arrow 2x. For example, in the absorbent-body continuous body 2, absorbent cores 4k containing pulp and/or superabsorbent polymer or the like are arranged in the first direction 2x with a spacing therebetween between two belt-form sheets 2a and 2b. The parts between the adjoining absorbent cores 4k of the absorbent-body continuous body 2 are cut, whereby the absorbent bodies 4 are formed.

The folding drum 21 is structured similarly to the folding device disclosed in Japanese Patent No. 2851784. The folding drum 21 is provided with a plurality of suction units 22 that move in the direction indicated by the arrow 25 along the cylindrical conveyance path 28. The suction units 22 each have a pair of suction tables 23 and 24 that open and close while moving. The suction tables 23 and 24 have suction surfaces 23s and 24s. Although not shown, suction holes communicating with a negative pressure source are formed in the suction surfaces 23s and 24s. The absorbent-body continuous body 2 fed through the guide roll 26 is sucked to the suction surfaces 23s and 24s of the opened suction surfaces 23 and 24, and conveyed.

The cutter roll 27 cuts the absorbent-body continuous body 2 between the adjoining suction units 22 when the parts between the adjoining suction units 22 of the absorbent-body continuous body 2 pass a facing position 80 where the cutter roll 27 and the folding drum 21 face each other, thereby forming the absorbent bodies 4. A first region 4f of each of the absorbent bodies 4 on the front side in the conveyance direction is sucked to the suction surface 23s of preceding one suction table 23 of the suction unit 22, and a second region 4b of each of the absorbent bodies 4 on the back side in the conveyance direction is sucked to the suction surface 24s of the other suction table 24 of the suction unit 22.

While sucking and conveying the absorbent body 4, the suction units 22 each sandwich the absorbent body 4 between the suction tables 23 and 24 by the suction tables 23 and 24 closing so as to face each other. Thereby, the absorbent body 4 is folded into two parts so as to form a fold line 4x orthogonal to the conveyance direction of the absorbent body 4 in which the absorbent body 4 is conveyed by the suction unit 22, whereby the folded absorbent body 5 is formed. The folded absorbent body 5 is divided into the first region 4f and the second region 4b by the fold line 4x.

After forming the folded absorbent body 5, the suction units 22 each make one suction table 23 stop sucking so that only the other suction table 24 sucks the folded absorbent body 5, and returns to the state where the suction tables 23 and 24 are open while moving along the cylindrical conveyance path 28. Then, when the other suction table 24 passes the transfer position facing the first repitching unit 30, the other suction table 24 stops sucking to transfer the folded absorbent body 5 to the first repitching unit 30.

The first repitching unit 30 has a first moving pad 32 that moves along a cylindrical conveyance path 38 while changing the speed. The first moving pad 32 has a suction surface 34 where suction holes communicating with a non-illustrated negative pressure source are formed. While moving at a first reception speed, the first moving pad 32 sucks, to the suction surface 34, the folded absorbent body 5 sucked to the suction table 24 of the folding drum 21 of the first unit 20, receives the folded absorbent body 5 from the folding drum 21, and while moving at a first transfer speed different from the first reception speed, transfers the received folded absorbent body 5 to the rotating unit 40. Structuring the first moving pad 32 so as to be moved by using a servo motor makes it easy to change the movement speed of the first moving pad 32.

The rotating unit 40 has a rotating pad 42 that rotates 90 degrees while moving along a cylindrical conveyance path 48. The rotating pad 42 has a suction surface 44 where suction holes communicating with a non-illustrated negative pressure source are formed, and receives the folded absorbent body 5 from the first repitching unit 30 by sucking, to the suction surface 44, the folded absorbent body 5 sucked to the first moving pad 32 of the first repitching unit 30. At this time, the rotating pad 42 receives the folded absorbent body 5 so that the fold line 4x of the folded absorbent body 5 is at the front. While conveying the folded absorbent body 5 while sucking it to the suction surface 44, the rotating pad 42 makes the fold line 4x of the folded absorbent body 5 parallel to the conveyance direction of the folded absorbent body 5 by rotating the folded absorbent body 5 by 90 degrees about a rotation axis extending in the normal direction of the rotating unit 40, and transfers the folded absorbent body 5 to the second repitching unit 50.

The second repitching unit 50 has a second moving pad 52 that moves along a cylindrical conveyance path 58 while changing the speed, and the second moving pad 52 has a suction surface 54 where suction holes communicating with a non-illustrated negative pressure source are formed. While moving at a second reception speed, the second moving pad 52 receives, from the rotating unit 40, the folded absorbent body 5 sucked to the rotating pad 42 of the rotating unit 40 and rotated by 90 degrees, by sucking the 90-degree rotated folded absorbent body 5 to the suction surface 54, and while moving at a second transfer speed different from the second reception speed, transfers the received folded absorbent body 5 to the second unit 60. Structuring the second moving pad 52 so as to be moved by a servo motor makes it easy to change the movement speed of the second moving pad 52.

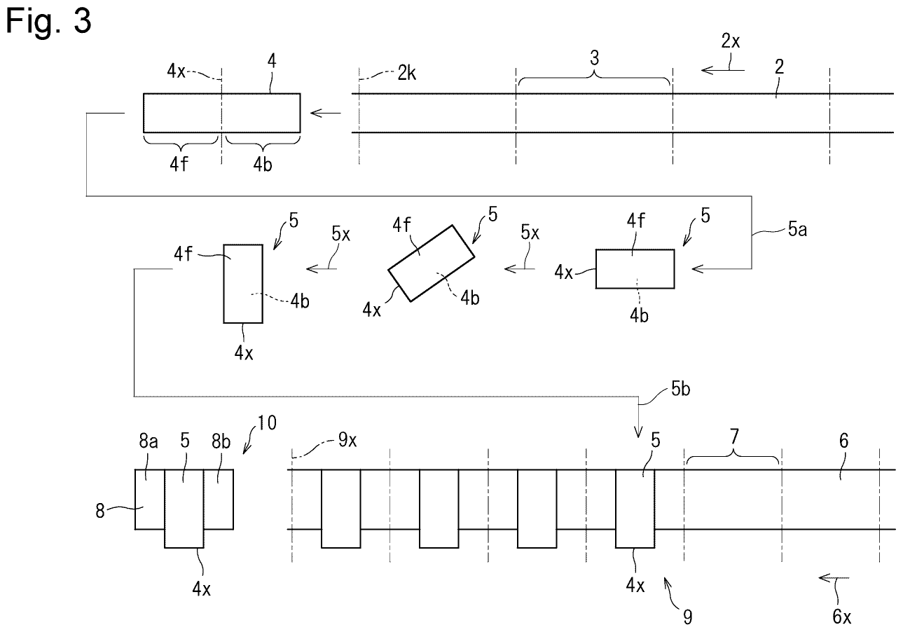

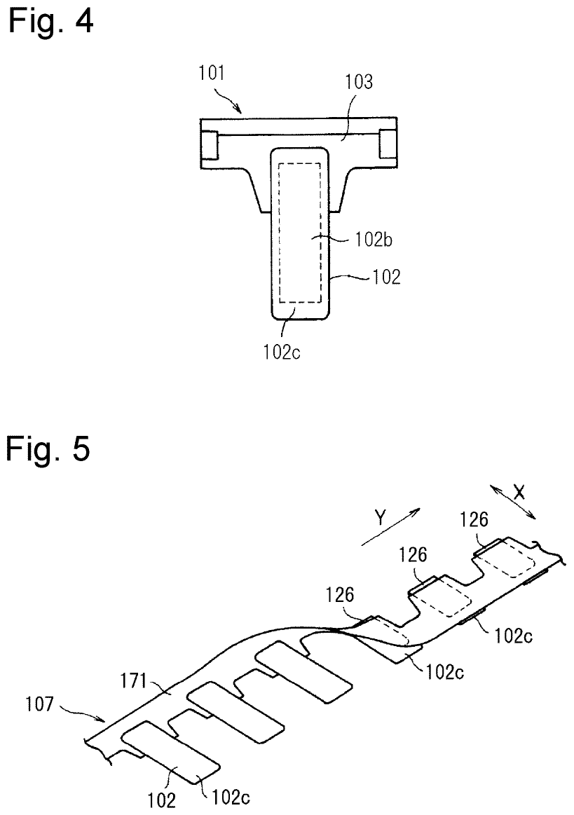

The second unit 60 has a conveyer 62 that conveys a waist-member continuous body 6, and an adhesive applier and a cutter which are not shown. In the waist-member continuous body 6, second portions 7 (see FIG. 3) to be made into waist members 8 (see FIG. 3) are connected in a second direction 6x.

The conveyer 62 is provided with an endless belt 64 having air permeability and circulating along a predetermined path and a non-illustrated suction box disposed inside the endless belt 64 and connected to a negative pressure source, and is structured so as to convey the waist-member continuous body 6 while sucking it to the outside of the endless belt 64.

The conveyer 62 is disposed adjacent to the second repitching unit 50, and is structured so that the 90-degree rotated folded absorbent bodies 5 from the second repitching unit 50 are placed on the waist-member continuous body 6 being conveyed by the conveyer 62, with a spacing therebetween.

The non-illustrated applier previously applies an adhesive to one or both of the overlapping parts of the waist-member continuous body 6 and the 90-degree rotated folded absorbent bodies 5.

When the 90-degree rotated folded absorbent bodies 5 are placed on the waist-member continuous body 6, they are bonded together to form a wear continuous body 9. The wear continuous body 9 is conveyed by the conveyer 62. The folded absorbent bodies 5 are bonded to the waist-member continuous body 6 in a state of being placed on the waist-member continuous body 6 so as to be along the waist-member continuous body 6 with the fold line 4x protruding from the waist-member continuous body 6. Instead of using an adhesive, the folded absorbent bodies 5 and the waist-member continuous body 6 may be bonded together by heat sealing, ultrasonic wave sealing or the like with the folded absorbent bodies 5 being placed on the waist-member continuous body 6.

The non-illustrated cutter cuts the wear continuous body 9 at cutting positions 9x (see FIG. 3) between the adjoining folded absorbent bodies 5 of the wear continuous body 9, thereby forming disposable wears 10 (see FIG. 3) where the folded absorbent body 5 is bonded at an intermediate position of the waist member 8 (see FIG. 3).

The temporarily fixing device 70 temporarily fixes together the first region 4f and the second region 4b divided by the fold line 4x of each folded absorbent body 5. That is, the bonding between the first region 4f and the second region 4b is made with a strength that can be released when the disposable wear 10 is used.

The temporarily fixing device 70 applies an adhesive to appropriate positions of the absorbent bodies 4 before being folded into two parts. The temporarily fixing device 70 may be structured so as to apply double-sided tape to appropriate positions of the absorbent bodies 4 before being folded into two parts or may be structured so as to temporarily fix appropriate positions of the folded absorbent bodies 5 after being folded into two parts, by bonding by heat sealing or ultrasonic wave sealing.

While the temporarily fixing device 70 may be omitted, performing temporarily fixing with the temporarily fixing device 70 being provided stabilizes the folded state and makes it possible to convey the folded absorbent bodies 5 at higher speed, so that the number of disposable wears 10 manufactured per unit time can be increased.

While the first repitching unit 30 may be omitted, the provision of the first repitching unit 30 between the first unit 20 and the rotating unit 40 makes it possible to accommodate size differences of the absorbent bodies 4.

For example, in a case where the size of the absorbent bodies 4 in the first direction 2x, that is, the predetermined length L is different, to manufacture the disposable wears 10 in the same cycle time, that is, to make the same the number of disposable wears 10 manufactured per unit time, if the speed at which the folded absorbent bodies 5 are conveyed at the rotating unit 40 is made the same, it is necessary to change the speed at which the absorbent-body continuous body 2 and the absorbent bodies 4 are conveyed at the first unit 20. Moreover, even if the size of the absorbent bodies 4 in the first direction 2x (predetermined length L) is the same, when the size of the absorbent bodies 4 in the width direction orthogonal to the first direction 2x is different, to manufacture the disposable wears 10 in the same cycle time, if the speed at which the first unit 20 conveys the absorbent-body continuous body 2 and the absorbent bodies 4 is made the same, it is necessary to change the speed at which the folded absorbent bodies 5 are conveyed at the rotating unit 40. As described above, when the conveyance speed of the folded absorbent bodies 5 at the first unit 20 and the conveyance speed of the folded absorbent bodies 5 at the rotating unit 40 are not the same, the first repitching unit 30 can stably convey the folded absorbent bodies 5 from the first unit 20 to the rotating unit 40. Consequently, the size differences of the absorbent bodies 4 can be accommodated.

Moreover, the provision of the first repitching unit 30 between the first unit 20 and the rotating unit 40 makes it possible to perform the folding of the absorbent bodies 4 and the rotating of the folded absorbent body 5 in an upward-facing state where bonding is more stabilized. This allows the absorbent bodies 4 and the folded absorbent bodies 5 to be conveyed at higher speed, which enables more efficient manufacture of the disposable wears 10.

While the second repitching unit 50 may be omitted, the provision of the second repitching unit 50 between the rotating unit 40 and the second unit 60 makes it possible to accommodate size differences of the waist members 8.

For example, when the size of the waist members 8 in the second direction 6x is different, to manufacture the disposable wears 10 in the same cycle time, if the conveyance speed of the 90-degree rotated folded absorbent bodies 5 at the rotating unit 40 is maintained the same, it is necessary to change the conveyance speed of the waist-member continuous body 6 at the second unit 60. In such a case, the second repitching unit 50 can change the conveyance speed of the 90-degree rotated folded absorbent bodies 5 when they are placed on the waist-member continuous body 6, so as to coincide with the conveyance speed of the waist-member continuous body 6. This makes it possible to accommodate size differences of the waist members.

The provision of both of the first repitching unit 30 and the second repitching unit 50 makes it possible to accommodate size differences of the disposable wears 10 even if the sizes of both of the absorbent bodies 4 and the waist members 8 change according to the size of the disposable wears 10.

In the disposable wear manufacturing apparatus 12, since the folding drum 21 of the first unit 20 folds the absorbent bodies 4 into two parts while conveying them along the cylindrical conveyance path 28, the section where the absorbent bodies 4 are to be folded into two parts can be made short compared with when the absorbent bodies 4 are folded into two parts while being conveyed along a linear conveyance path.

Next, a method for manufacturing the disposable wear 10 will be described with reference to FIG. 3. FIG. 3 is an explanatory view showing a disposable wear manufacturing process. As shown in FIG. 3, the disposable wear 10 is formed by using the absorbent-body continuous body 2 and the waist-member continuous body 6.

The method for manufacturing the disposable wear 10 is provided with the following steps (1) to (7):

### (1) First Conveying Step

The absorbent-body continuous body 2 is conveyed in the first direction 2x indicated by the arrow 2x. In the absorbent-body continuous body 2, the first portions 3 to be made into the absorbent bodies 4 are connected in the first direction 2x. The absorbent bodies 4 may be continuously formed and conveyed, or previously prepared absorbent bodies 4 may be conveyed.

### (2) Second conveying step

The waist-member continuous body 6 is conveyed in the second direction 6x indicated by the arrow 6x. In the waist-member continuous body 6, the second portions 7 to be made into the waist members 8 are connected in the second direction 6x. The waist-member continuous body 6 may be continuously formed and conveyed, or a previously prepared waist-member continuous body 6 may be conveyed.

### (3) First Cutting Step

The absorbent-body continuous body 2 being conveyed in the first direction 2x is cut at the cutting positions indicated by a chain line 2k, thereby forming the absorbent bodies 4.

### (4) Folding Step

As shown by the arrow 5a, while being conveyed in the cylindrical conveyance path 28 (see FIG. 1), the cut absorbent bodies 4 are each folded into two parts at an intermediate position of the absorbent body 4 in the first direction 2x so that the fold line 4x orthogonal to the first direction 2x is formed, thereby forming the folded absorbent bodies 5. The folded absorbent bodies 5 are each divided into the first region 4f and the second region 4b by the fold line 4x, and the first region 4f and the second region 4b overlap each other.

### (5) Rotating Step

The folded absorbent bodies 5 are rotated by 90 degrees while being conveyed in the direction indicated by the arrow 5x. That is, the direction of the fold line 4x of each folded absorbent body 5 relative to the conveyance direction 5x is changed by 90 degrees so that the conveyance direction 5x of the folded absorbent bodies 5 and the fold line 4x are parallel to each other.

### (6) Bonding Step

As shown by the arrow 5b, the 90-degree rotated folded absorbent bodies 5 are placed on the waist-member continuous body 6 being conveyed in the second direction 6x, and bonded thereto. That is, a plurality of 90-degree rotated folded absorbent bodies 5 are placed on the waist-member continuous body 6 being conveyed in the second direction 6x, with a spacing therebetween, and the folded absorbent bodies 5 are bonded to the waist-member continuous body 6 to form the wear continuous body 9. At this time, the folded absorbent bodies 5 are placed on the waist-member continuous body 6 so as to be along the waist-member continuous body 6 with the fold lines 4x of the folded absorbent bodies 5 protruding from the waist-member continuous body 6, and the part of each folded absorbent body 5 facing the waist-member continuous body 6 is bonded to the waist-member continuous body 6. For example, the folded absorbent bodies 5 and the waist-member continuous body 6 are bonded together by previously applying an adhesive to appropriate positions of the folded absorbent bodies 5 and/or the waist-member continuous body 6 or by heat sealing, ultrasonic wave sealing or the like with the folded absorbent bodies 5 being placed on the waist-member continuous body 6.

### (7) Second Cutting Step

The wear continuous body 9 is cut at the cutting positions 9x indicated by the chain line 9x between the adjoining folded absorbent bodies 5, thereby forming the disposable wears 10 where the folded absorbent body 5 is bonded to an intermediate position of the waist member 8.

To the waist member 8, one of the first and second regions 4f and 4b of the folded absorbent body 5 is bonded. A non-illustrated hook-and-loop fastener for bonding to the other of the first and second regions 4f and 4b of the folded absorbent body 5 may be provided on parts 8a and 8b near both ends of the waist member 8. This hook-and-loop fastener may be previously provided on the waist-member continuous body 6, or may be provided on the disposable wears 10 after the cutting. Other accessory members may be previously provided on the absorbent-body continuous body 2 and/or the waist-member continuous body 6, or may be provided on the disposable wears 10 after the cutting.

When the disposable wears 10 are formed by the above-described steps (1) to (7), since the absorbent bodies 4 are folded into two parts while being conveyed along the cylindrical conveyance path, the section where the absorbent bodies 4 are to be folded into two parts can be made short compared with when the absorbent bodies 4 are folded into two parts while being conveyed along a linear conveyance path.

The method for manufacturing the disposable wear 10 may be further provided with any one or more of the following steps (8) to (10).

### (8) Temporarily Fixing Step

The first region 4f and the second region 4b of the folded absorbent body 5 are temporarily fixed together. For example, they are temporarily fixed by applying an adhesive or applying double-sided tape to appropriate positions of the absorbent body 4 before folded into two parts. The temporary fixing may be performed by temporarily fixing appropriate positions of the folded absorbent body 5 by heat sealing, ultrasonic wave sealing or the like after the absorbent body 4 is folded into two parts. The folded absorbent body 5 having undergone the temporary fixing can be conveyed at higher speed because its folded state is stabilized, so that the number of disposable wears manufactured per unit time can be increased.

### (9) First Repitching Step

By using the first moving pad 32 (see FIG. 1), the folded absorbent bodies 5 are received from the folding step, and the received folded absorbent bodies 5 are transferred to the rotating step. The first moving pad 32 receives the folded absorbent bodies 5 from the folding step while moving at the first reception speed, and transfers the received folded absorbent bodies 5 to the rotating step while moving at the first transfer speed different from the first reception speed. The first repitching step makes it possible to accommodate size differences of the absorbent bodies.

### (10) Second Repitching Step

By using the second moving pad 52 (see FIG. 2), the 90-degree rotated folded absorbent bodies 5 are received from the rotating step, and the received folded absorbent bodies 5 are transferred to the bonding step. The second moving pad 52 receives the 90-degree rotated folded absorbent bodies 5 from the rotating step while moving at the second reception speed, and transfers the received folded absorbent bodies 5 to the bonding step while moving at the second transfer speed different from the second reception speed. The second repitching step makes it possible to accommodate size difference of the waist members.

The provision of both of the first repitching step and the second repitching step makes it possible to accommodate size differences of the disposable wears 10 even if the sizes of both of the absorbent bodies 4 and the waist members 8 change according to the size of the disposable wears 10.

[Summary] As described above, by folding the absorbent bodies into two parts while conveying them along the cylindrical conveyance path, the section where the absorbent bodies are to be folded into two parts can be shortened.

The present invention is not limited to the above-described embodiment but may be carried out with various modifications being added.

### DESCRIPTION OF REFERENCE NUMERALS

2 Absorbent-body continuous body
2x First direction
3 First portion
4 Absorbent body
4b Second region
4f First region
4x Fold line
5 Folded absorbent body
6 Waist-member continuous body
6x Second direction
7 Second portion
8 Waist member
9 Wear continuous body
9x Cutting position
10 Disposable wear
12 Disposable wear manufacturing apparatus
20 First unit
28 Cylindrical conveyance path
30 First repitching unit
32 First moving pad
40 Rotating unit
50 Second repitching unit
52 Second moving pad
60 Second unit
70 Temporarily fixing device

1-10. (canceled)

11. A disposable wear manufacturing method comprising:
a first conveying step of conveying, in a first direction, an absorbent-body continuous body in which first portions to be made into absorbent bodies are connected in the first direction;
a second conveying step of conveying, in a second direction, a waist-member continuous body in which second portions to be made into waist members are connected in the second direction;
a first cutting step of forming the absorbent bodies by cutting the absorbent-body continuous body being conveyed in the first direction;
a folding step of forming folded absorbent bodies by folding the cut absorbent bodies into two parts so that fold lines are formed in a direction orthogonal to the first direction while conveying the cut absorbent bodies along a cylindrical conveyance path;
a rotating step of rotating the folded absorbent bodies by 90 degrees while conveying the folded absorbent bodies;
a bonding step of forming a wear continuous body by placing more than one of the 90-degree rotated absorbent bodies on the waist-member continuous body being conveyed in the second direction, with a spacing therebetween, and bonding the absorbent bodies to the waist-member continuous body; and
a second cutting step of forming disposable wears in each of which the folded absorbent body is bonded to an intermediate position of the waist member, by cutting the wear continuous body at cutting positions between the adjoining absorbent bodies.

12. The disposable wear manufacturing method according to claim 11, further comprising
a temporarily fixing step of temporarily fixing together a first region and a second region divided by the fold line of each of the folded absorbent bodies.

13. The disposable wear manufacturing method according to claim 11, further comprising
a first repitching step where by using a first moving pad, the folded absorbent bodies are received from the folding step, the received folded absorbent bodies are transferred to the rotating step, and the first moving pad receives the folded absorbent bodies from the folding step while moving at a first reception speed and transfers the received folded absorbent bodies to the rotating step while moving at a first transfer speed different from the first reception speed.

14. The disposable wear manufacturing method according to claim 12, further comprising
a first repitching step where by using a first moving pad, the folded absorbent bodies are received from the folding step, the received folded absorbent bodies are transferred to the rotating step, and the first moving pad receives the folded absorbent bodies from the folding step while moving at a first reception speed and transfers the received folded absorbent bodies to the rotating step while moving at a first transfer speed different from the first reception speed.

15. The disposable wear manufacturing method according to claim 11, further comprising
a second repitching step where by using a second moving pad, the 90-degree rotated folded absorbent bodies are received from the rotating step, the received folded absorbent bodies are transferred to the bonding step, and the second moving pad receives the 90-degree rotated folded absorbent bodies from the rotating step while moving at a second reception speed and transfers the received folded absorbent bodies to the bonding step while moving at a second transfer speed different from the second reception speed.

16. The disposable wear manufacturing method according to claim 12, further comprising
a second repitching step where by using a second moving pad, the 90-degree rotated folded absorbent bodies are received from the rotating step, the received folded absorbent bodies are transferred to the bonding step, and the second moving pad receives the 90-degree rotated folded absorbent bodies from the rotating step while moving at a second reception speed and transfers the received folded absorbent bodies to the bonding step while moving at a second transfer speed different from the second reception speed.

17. The disposable wear manufacturing method according to claim 11, further comprising:
a first repitching step where by using a first moving pad, the folded absorbent bodies are received from the folding step, the received folded absorbent bodies are transferred to the rotating step, and the first moving pad receives the folded absorbent bodies from the folding step while moving at a first reception speed and transfers the received folded absorbent bodies to the rotating step while moving at a first transfer speed different from the first reception speed; and
a second repitching step where by using a second moving pad, the 90-degree rotated folded absorbent bodies are received from the rotating step, the received folded absorbent bodies are transferred to the bonding step, and the second moving pad receives the 90-degree rotated folded absorbent bodies from the rotating step while moving at a second reception speed and transfers the received folded absorbent bodies to the bonding step while moving at a second transfer speed different from the second reception speed.

18. The disposable wear manufacturing method according to claim 12, further comprising:
a first repitching step where by using a first moving pad, the folded absorbent bodies are received from the folding step, the received folded absorbent bodies are transferred to the rotating step, and the first moving pad receives the folded absorbent bodies from the folding step while moving at a first reception speed and transfers the received folded absorbent bodies to the rotating step while moving at a first transfer speed different from the first reception speed; and
a second repitching step where by using a second moving pad, the 90-degree rotated folded absorbent bodies are received from the rotating step, the received folded absorbent bodies are transferred to the bonding step, and the second moving pad receives the 90-degree rotated folded absorbent bodies from the rotating step while moving at a second reception speed and transfers the received folded absorbent bodies to the bonding step while moving at a second transfer speed different from the second reception speed.

19. A disposable wear manufacturing apparatus comprising:
a first unit that forms absorbent bodies by cutting an absorbent-body continuous body in which first portions to be made into the absorbent bodies are connected in a first direction, while conveying the absorbent-body continuous body in the first direction, and forms folded absorbent bodies by folding the absorbent bodies into two parts so that fold lines are formed in a direction orthogonal to a conveyance direction of the absorbent bodies, while conveying the absorbent bodies along a cylindrical conveyance path;
a rotating unit that rotates the folded absorbent bodies by 90 degrees while conveying the folded absorbent bodies; and
a second unit that forms a wear continuous body by placing more than one of the 90-degree rotated folded absorbent bodies on a waist member continuous body in which second portions to be made into waist members are connected in a second direction, with a spacing therebetween while conveying the waist member continuous body in the second direction and bonding the folded absorbent bodies to the waist-member continuous body, and forms disposable wears in each of which the folded absorbent body is bonded to an intermediate position of the waist member, by cutting the wear continuous body at cutting positions between the adjoining absorbent bodies.

20. The disposable wear manufacturing apparatus according to claim 19, further comprising
a temporarily fixing device that temporarily fixes together a first region and a second region divided by the fold line of each of the folded absorbent bodies.

21. The disposable wear manufacturing apparatus according to claim 19, further comprising
a first repitching unit having a first moving pad that receives the folded absorbent bodies from the first unit while moving at a first reception speed and transfers the received folded absorbent bodies to the rotating unit while moving at a first transfer speed different from the first reception speed.

22. The disposable wear manufacturing apparatus according to claim 20, further comprising
a first repitching unit having a first moving pad that receives the folded absorbent bodies from the first unit while moving at a first reception speed and transfers the received folded absorbent bodies to the rotating unit while moving at a first transfer speed different from the first reception speed.

23. The disposable wear manufacturing apparatus according to claim 19, further comprising
a second repitching unit having a second moving pad that receives the 90-degree rotated folded absorbent bodies from the rotating unit while moving at a second reception speed and transfers the received absorbent bodies to the second unit while moving at a second transfer speed different from the second reception speed.

24. The disposable wear manufacturing apparatus according to claim 20, further comprising
a second repitching unit having a second moving pad that receives the 90-degree rotated folded absorbent bodies from the rotating unit while moving at a second reception speed and transfers the received absorbent bodies to the second unit while moving at a second transfer speed different from the second reception speed.

25. The disposable wear manufacturing apparatus according to claim 19, further comprising:
a first repitching unit having a first moving pad that receives the folded absorbent bodies from the first unit while moving at a first reception speed and transfers the received folded absorbent bodies to the rotating unit while moving at a first transfer speed different from the first reception speed; and
a second repitching unit having a second moving pad that receives the 90-degree rotated folded absorbent bodies from the rotating unit while moving at a second reception speed and transfers the received absorbent bodies to the second unit while moving at a second transfer speed different from the second reception speed.

26. The disposable wear manufacturing apparatus according to claim 20, further comprising:
a first repitching unit having a first moving pad that receives the folded absorbent bodies from the first unit while moving at a first reception speed and transfers the received folded absorbent bodies to the rotating unit while moving at a first transfer speed different from the first reception speed; and
a second repitching unit having a second moving pad that receives the 90-degree rotated folded absorbent bodies from the rotating unit while moving at a second reception speed and transfers the received absorbent bodies to the second unit while moving at a second transfer speed different from the second reception speed.
